(19) United States
(12) Patent Application Publication
(10) Pub. No.: US 2011/0034937 A1
(43) Pub. Date: Feb. 10, 2011

(54) Revascularization Device for Treating an Occluded Arterial Vessel

(75) Inventors: Jihad Mustapha, Ada, MI (US); Tony Das, Dallas, TX (US)

Correspondence Address: Jonathan P. O''Brien, Ph.D.;Honigman Miller Schwartz and Cohn LLP, 350 East Michigan Avenue, Suite 300, KALAMAZOO, MI, 49007, US

(73) Assignee: TD.JAM Medical Technologies, LLC, Ada, MI (US)

(21) Appl. No.: 12/851,867

(22) Filed: Aug. 6, 2010

Related U.S. Application Data
(60) Provisional application No. 61/232,155, filed on Aug. 7, 2009.

(51) Int. Cl. A61B 17/22

(52) U.S. Cl. 606/127

(57) ABSTRACT

Medical devices for revascularization and methods of their use are disclosed. The medical device includes an actuator portion, an inner tubular body connected to the actuator portion and an outer tubular body connected to the actuator portion. The inner tubular body is disposed within a bore of the outer tubular body. The medical device also includes a guide wire extending through one or more of the actuator portion, inner tubular body and outer tubular body.

This application claims the benefit of U.S. Provisional Application No. 61/232,155, filed on Aug. 7, 2009, which is herein incorporated by reference.

### FIELD OF THE INVENTION

The disclosure relates to medical devices and to a device for revascularizing a totally occluded or partially occluded vessel.

### DESCRIPTION OF THE RELATED ART

Modern medical technology has realized many benefits for mankind. For example, modern medical technology has permitted medical professionals to treat their patients with novel, relatively low-risk, minimally invasive procedures instead of conventional surgical techniques. As a result, modern medical technology has permitted medical professionals to successfully prolong the life of many patients while also reducing hospital stays and other costs associated with the treatment of a patient.

Although many benefits have been realized by utilizing modern medical technology, such technology is nevertheless susceptible to improvements that may enhance and/or advance patient treatment while also reducing associated costs, risks and the like. Therefore, a need exists in the art for the development of improved medical technology that includes but is not limited to medical devices and methods for using medical devices.

### SUMMARY

Medical devices are disclosed, which may, in some embodiments of the present disclosure, include an actuator portion; an inner tubular body connected to the actuator portion; an outer tubular body connected to the actuator portion, wherein the inner tubular body is disposed within a bore of the outer tubular body; and a guide wire extending through one or more of the actuator portion, inner tubular body and outer tubular body. In some embodiments, the actuator portion may provide means for manipulating an orientation of the inner tubular body relative to one or more of the outer tubular body and guide wire for penetrating a blockage within a vessel for revascularizing the vessel. In some embodiments, the manipulation of the orientation of the inner tubular body may be a single, forceful plunging movement through the blockage, where the blockage includes a chronic total occlusion or partial occlusion, and wherein the vessel may be an artery.

In some embodiments, the inner tubular body may include a distal end that may provide means for penetrating a blockage within a vessel for revascularizing the vessel in response to a manipulation of the actuator portion. In some embodiments, the penetrating of the blockage within the vessel is conducted by a single, forceful plunging movement of the inner tubular body through the blockage, where the blockage may include a chronic total occlusion or partial occlusion, and where the vessel may be an artery. In some embodiments, a bore of the inner tubular body may be connected to an aspiration device. In some embodiments, a distal end of the inner tubular body may include a piercing portion. In some embodiments, the piercing portion may form an opening at the distal end of the inner tubular body, and a bore of the inner tubular body may be connected to an aspiration device for aspirating a portion of the blockage out of the vessel.

In some embodiments, the actuator portion may include a deployment actuator, a selectively releasable fastener connected to the deployment actuator, a deployment actuator reset member connectable with the selectively releasable fastener, and an energy storage member connected to the deployment actuator reset member. In some embodiments, the deployment actuator member may include a depressible button, where the selectively releasable fastener includes a latch, where the deployment actuator reset member includes a lever, and where the energy storage member includes a spring. In some embodiments, the distal end of the inner tubular body may further comprise a piercing portion. In some embodiments, an inflatable portion may be connected to an outer surface of the outer tubular body.

In some embodiments of the present disclosure, a method of revascularizing a totally occluded or partially occluded vessel containing at least one blockage may include providing a revascularization device which may include an actuator portion; an inner tubular body connected to the actuator portion, wherein the inner tubular body comprises a distal end; an outer tubular body connected to the actuator portion, wherein the inner tubular body is disposed within a bore of the outer tubular body; and a guide wire extending through one or more of the actuator portion, inner tubular body and outer tubular body. Some embodiments of the disclosed methods may include locating the distal end of the inner tubular body proximate to the at least one blockage in the vessel and manipulating the actuator portion so that the distal end of the inner tubular body penetrates the blockage. In some embodiments of the disclosed methods, the revascularization device may further include an inflatable portion, and the method may include inflating the inflatable portion.

In some embodiments of the present disclosure, methods of revascularizing a totally occluded or partially occluded vessel may include providing a revascularization device, which may include an actuator portion, an inner tubular body detachably connected to the actuator portion, where the inner tubular body comprises a distal end; manipulating the actuator portion so that the distal end of the inner tubular body penetrates the blockage, creating a bore through the blockage; detaching the inner tubular body; and attaching a medical interventional device to the actuator portion. In some embodiments of the disclosed methods, the medical interventional device may comprise a stent attached thereto; and the method may further include placing the stent at least partially within the bore through the blockage by manipulating the actuator portion. In some embodiments of the disclosed method, the revascularization device may further include an inflatable portion, and the method may further include inflating the inflatable portion.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

The embodiments of the present disclosure will now be described, by way of example, with reference to the accompanying drawings in which:

FIG. 1A is a side view of an example embodiment of a revascularization device of the present disclosure including a perspective view of the distal end of the revascularization device and a longitudinal-sectional view of the proximal end of the revascularization device;

FIG. 1B is another side view of the revascularization device depicted in FIG. 1A, illustrating a manipulated orientation of both of the proximal end and the distal end in accordance with an example embodiment of the present disclosure; and

FIGS. 2A-2D illustrate a plurality of longitudinal-sectional views of the distal end of an example embodiment of a revascularization device of the present disclosure during a procedure to create a bore through a blockage in a totally occluded arterial vessel.

FIGS. 3A-3D illustrate a plurality of longitudinal-sectional views of the distal end of an example embodiment of a revascularization device of the present disclosure during a procedure to insert a stent into a bore through a blockage in a totally occluded arterial vessel.

### DETAILED DESCRIPTION OF THE INVENTION

The Figures illustrate example embodiments of an apparatus associated with revascularization treatment of totally occluded or partially occluded vessels. Based on the foregoing, it is to be generally understood that the nomenclature used herein is simply for convenience and the terms used to describe the invention should be given the broadest meaning by one of ordinary skill in the art.

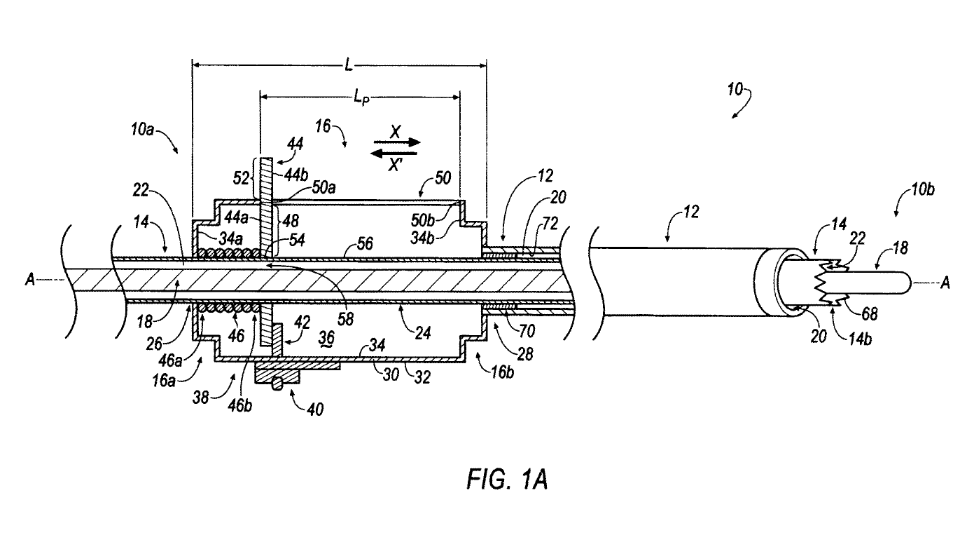

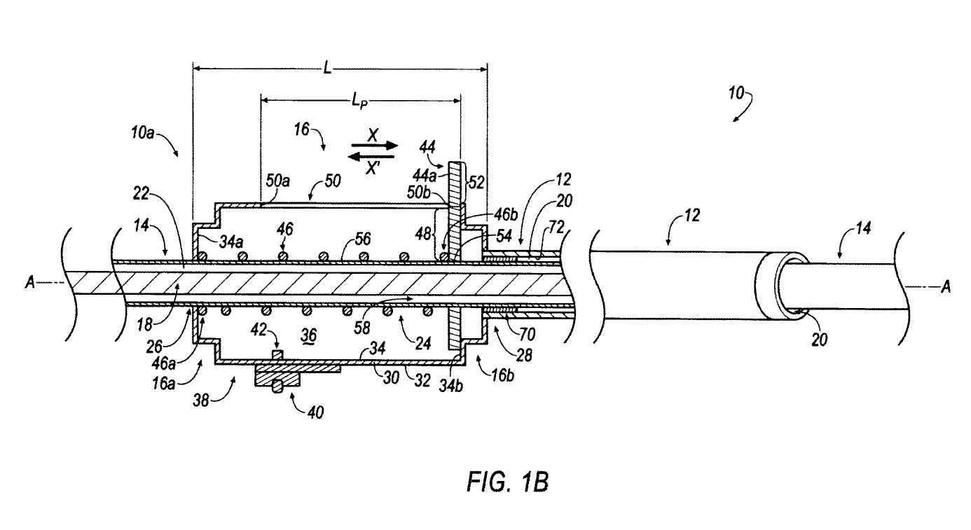

Referring to FIGS. 1A and 1B, a revascularization device is shown generally at 10 in accordance with an example embodiment of the present disclosure. Revascularization device 10 includes a proximal end 10a and a distal end 10b. In some embodiments, a user may manipulate an orientation of the proximal end 10a in order to affect a manipulation of an orientation of the distal end 10b. For example, device 10, shown in the undeployed state in FIG. 1, may be manipulated to yield the deployed orientation depicted in FIG. 1B. It will be appreciated that other orientations, for example, other intermediate orientations between a deployed and undeployed state and other deployed and undeployed orientations are possible.

In some embodiments of the present disclosure, one or more of the proximal and distal ends 10a, 10b of a revascularization device 10 may be formed by an outer tubular body 12, an inner tubular body 14, an actuator portion 16 and a guide wire 18. In some embodiments, inner tubular body 14 is arranged to be displaceable within a bore 20 of outer tubular body 12. In some embodiments, one or more guide wires 18 are arranged to be displaceable within a bore 22 of inner tubular body 14.

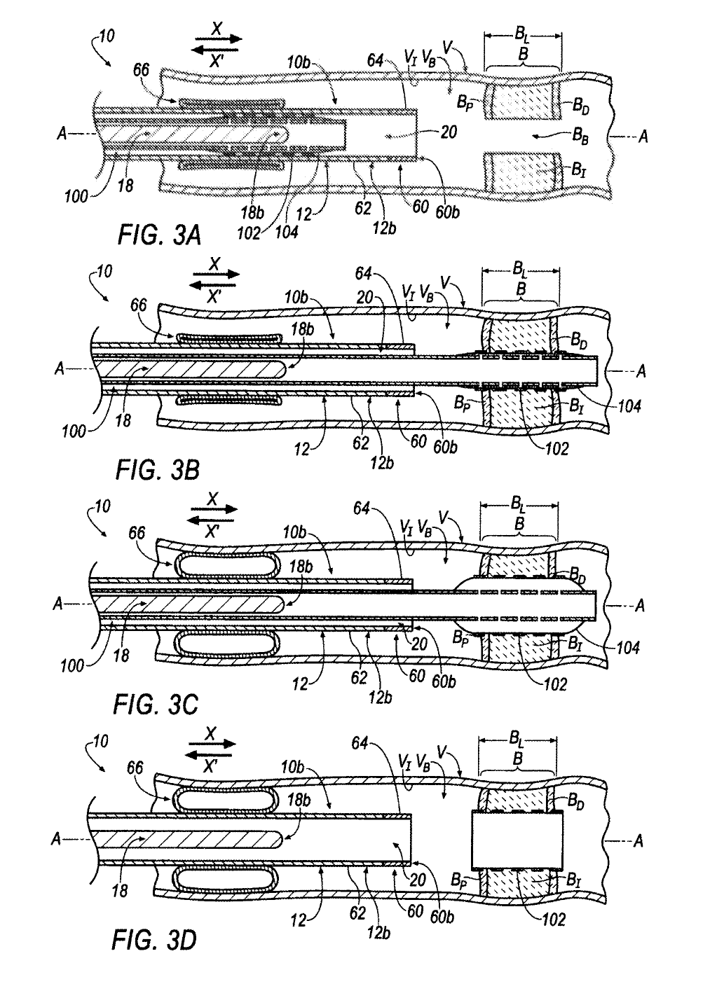

It will be appreciated that a guide wire 18 is not limited to being exclusively disposed within bore 22 of inner tubular body 14 and that guide wire 18 may be arranged within a bore of any particular body depending on a particular arrangement of the revascularization device 10. For example, in some embodiments inner tubular body 14 may be removed in the direction of the arrow X′ from within bore 20 of outer tubular body 12 such that inner tubular body 14 is disconnected from revascularization device 10, as described in more detail below (see also, e.g., FIGS. 3A-3D). Guide wire 18 may be located within bore 20 of outer tubular body 12 or within a bore of an alternative tubular body 100 (see FIGS. 3A-3C).

In FIGS. 1A and 1B, a reference axis, A-A, is shown extending through the revascularization device 10. In some embodiments, guide wire 18 may be centered about the axis, A-A, such that the axis, A-A, traverses an axial center of the guide wire 18. Further, in some embodiments, outer and inner tubular bodies 12, 14 are arranged about guide wire 18 such that outer and inner tubular bodies 12, 14 are also arranged about the axis A-A in a substantially concentric orientation relative to guide wire 18.

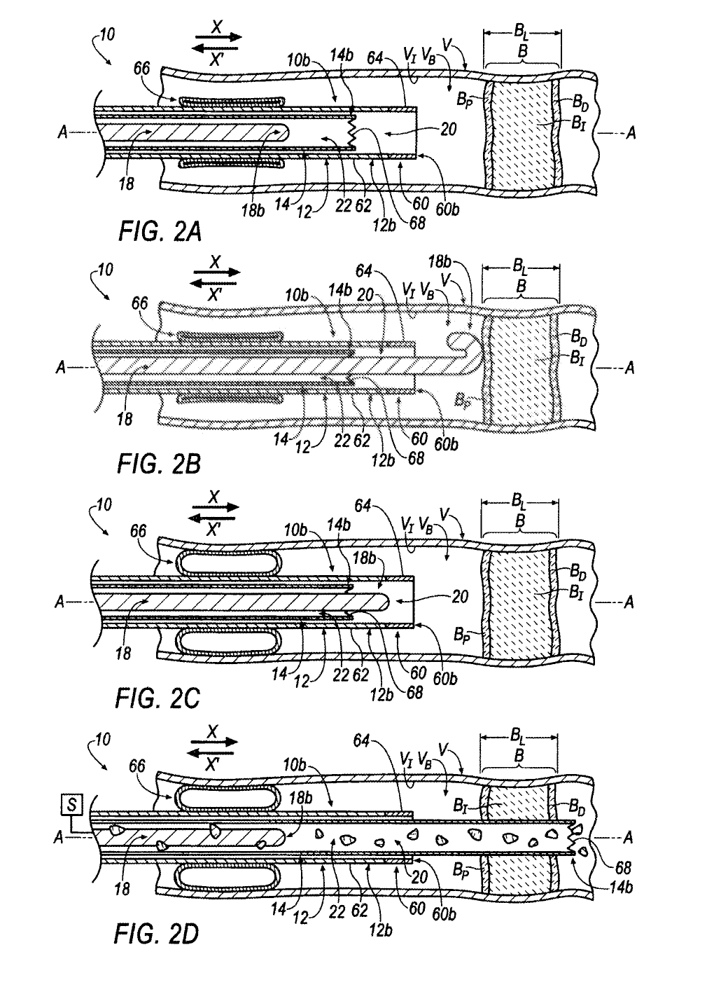

As provided in more detail below, at least a portion of the distal end 10b of revascularization device 10 may be inserted and/or disposed within a bore, V_{B}, of a vessel, V during a revascularization procedure (see, e.g., FIGS. 2A-3D). It will be appreciated, however, that when such a vessel, V, is, e.g., non-axial, tortuous, or serpentine, one or more portions of the distal end 10b of revascularization device 10 may not necessarily be co-axial with the axis, A-A, while the distal end 10b of revascularization device 10 is being navigated through the bore, V_{B}, of a vessel, V. However, it will be appreciated that in some embodiments, although the proximal end 10b of revascularization device 10 may be disposed within the bore, V_{B}, of a vessel, V, that may be, e.g., non-axial, tortuous, or serpentine, one or more of outer and inner tubular bodies 12, 14 may retain a substantially concentric relationship with guide wire 18.

With continued reference to FIGS. 1A and 1B, actuator portion 16 in some embodiments of revascularization device 10 may be located at or at least proximate to the proximal end 10a of revascularization device 10. In some embodiments, actuator portion 16 may include a proximal end 16a and a distal end 16b. In some embodiments, actuator portion 16 may include a bore 24 that may extend through an entire length, L, of actuator portion 16. In some embodiments, access to the bore 24 may be permitted by way of a proximal opening 26 formed at the proximal end 16a of actuator portion 16 and a distal opening 28 formed at the distal end 16b of actuator portion 16.

In some embodiments of the present disclosure, one or more of outer tubular body 12, inner tubular body 14 and guide wire 18 may be arranged within the bore 24 of actuator portion 16. In some embodiments, one or more of outer tubular body 12, inner tubular body 14 and guide wire 18 may extend axially beyond one or more of the proximal end 16a and the distal end 16b of actuator portion 16. In some embodiments, outer tubular body 12 may extend completely or partway within the bore 24 of actuator portion 16 such that outer tubular body 12 is connected to actuator portion 16 at least proximate to the distal opening 28 formed in the distal end 16b of actuator portion 16.

In some embodiments, actuator portion 16 may include a body 30 having an outer surface 32 and an inner surface 34. In some embodiments, inner surface 34 may further define a cavity 36, volume or the like within the body 30. In some embodiments, the cavity 36 defined by inner surface 34 may be partially contiguous with bore 24 or completely contiguous with bore 24, such that inner surface 34 defines a single cavity 36.

In some embodiments, actuator portion 16 may further comprise an actuator assembly 38 that is attached, arranged or otherwise disposed within or upon one or more of the outer surface 32, inner surface 34 and bore 24/cavity 36 of the actuator portion 16, as well as inner tubular body 14, as described below.

In some embodiments, actuator assembly 38 may include a deployment actuator 40, a selectively releasable fastener 42, a deployment actuator reset member 44 and an energy storage member 46. In some embodiments, deployment actuator member 40 may include a depressible button or the like. In some embodiments, selectively releasable fastener 42 may include a latch or the like. In some embodiments, deployment actuator reset member 44 may include a lever/handle or the like. In some embodiments, energy storage member 46 may include a spring or the like.

In some embodiments, deployment actuator 40 may be arranged upon or at least proximate to the outer surface 32 of the body 30 of the actuator portion 16. In some embodiments, selectively releasable fastener 42 may be arranged within bore 24/cavity 36 and upon or at least proximate to the inner surface 34 of body 30 of actuator portion 16. In some embodiments, deployment actuator 40 may be in direct or indirect communication with selectively releasable fastener 42, such that a user depressing or otherwise manipulating deployment actuator 40 results in a manipulation of the orientation of selectively releasable fastener 42 such that the orientation of selectively releasable fastener 42 is changed from being in a undeployed orientation (FIG. 1A) to an deployed orientation (FIG. 1B).

In some embodiments, energy storage member 46 and at least a first portion 48 of deployment actuator reset member 44 may be arranged within bore 24/cavity 36 of actuator portion 16. In some embodiments, a slot 50 or the like may be formed in the body 30 of actuator portion 16 such that at least a second portion 52 of deployment actuator reset member 44 may extend outside of bore 24/cavity 36 and at or beyond the outer surface 32 of actuator portion 16, such that it may be manipulable from the outside of the device by a user. It will be appreciated that slot 50 may include a passage, opening, channel or other suitable conformations to allow actuator reset member 44 to extend past the outer surface 32 of actuator portion 16.

In some embodiments, the inner surface 34 of body 30 of actuator portion 16 may further define an inner proximal surface 34a and an inner distal surface 34b. In some embodiments, energy storage member 46 may include a proximal end 46a and a distal end 46b. In some embodiments, deployment actuator reset member 44 may include a proximal end surface 44a and a distal end surface 44b.

In some embodiments, the proximal end 46a of energy storage member 46 may be arranged substantially adjacent to the inner proximal surface 34a of the inner surface 34 of body 30 of actuator portion 16. In some embodiments, the distal end 46b of energy storage member 46 may be arranged substantially adjacent to the proximal end surface 44a of deployment actuator reset member 44. Further, in some embodiments, energy storage member 46 may be disposed about and circumscribe an outer surface 56 of the inner tubular body 14.

In some embodiments, slot 50 formed in the body 30 of the actuator portion 16 may include proximal slot end surface 50a and a distal slot end surface 50b. In some embodiments, when selectively releasable fastener 42 is in an undeployed state (see, e.g., FIG. 1A), the proximal end surface 44a of deployment actuator reset member 44 may be disposed adjacent or at least proximate to the proximal slot end surface 50a of slot 50. When deployment actuator reset member 44 is arranged in the undeployed state as described immediately above, energy storage member 46 may be arranged in a compressed orientation in order to store potential energy.

In some embodiments, when selectively releasable fastener 42 is in a deployed state (see, e.g., FIG. 1B), the distal end surface 44b of deployment actuator reset member 44 may be disposed adjacent or at least proximate to the distal slot end surface 50b of slot 50. When deployment actuator reset member 44 is arranged as described immediately above, energy storage member 46 may be arranged in an expanded orientation such that the previously stored potential energy is/has been released.

Referring back to FIG. 1A, in some embodiments, upon selectively releasable fastener 42 being manipulated from the undeployed to the deployed state, the immediate release of the potential energy stored by energy storage member 46 imparts a force to deployment actuator reset member 44 in the direction of the arrow, X, in order to cause deployment actuator reset member 44 to travel a portion, L_{P}, of the length, L, of actuator portion 16. In some embodiments, the portion, L_{P}, of the length, L, of actuator portion 16 is approximately equal to the length of slot 50 formed in the body 30 of actuator portion 16.

In some embodiments, actuator assembly 38 may be in direct/indirect contact or be in communication with inner tubular body 14 to permit a manipulation of an orientation of inner tubular body 14 relative to one or more of outer tubular body 12 and guide wire 18. In some embodiments, at least a portion 54 of actuator assembly 38 is affixed to the outer surface 56 of inner tubular body 14. In some embodiments, portion 54 of the actuator assembly 38, which is affixed to at least a portion of the outer surface 56 of the inner tubular body 14, may include, for example, a surface of deployment actuator reset member 44. In some embodiments, at least a portion of the outer surface 56 of inner tubular body 14 may be detachably affixed to a portion 54, of actuator assembly 38. In some embodiments, at least a portion of the outer surface 56 of inner tubular body 14 may be detachably affixed to a surface of deployment actuator reset member 44. Because surface 54 of deployment actuator reset member 44 is attached to the outer surface 56 of inner tubular body 14, the travel of deployment actuator reset member 44 along the portion, L_{P}, of the length, L, of the actuator portion 16 in either the direction of arrow X or the arrow orientated in the opposite direction, X,′ results in a corresponding movement of inner tubular body 14. As such, inner tubular body 14 may be permitted to travel with a forceful, plunging movement relative to one or more of outer tubular body 12 and guide wire 18 according to the direction of the arrow X as a result of the stored potential energy of energy storage member 46 being released. In some embodiments, a user may manually move inner tubular body 14 back to the undeployed state in the direction of the arrow, X′, by grasping and moving, for example, deployment actuator reset member 44 back to its undeployed state. As shown, for example, in FIG. 1A, energy storage member 46 may be compressed and selectively releasable fastener 42 returned to its undeployed state for a subsequent, selective deployment of the inner tubular body 14.

In some embodiments, deployment actuator reset member 44 may be configured to include an interior surface 54, which may define a bore 58 or passage extending through deployment actuator reset member 44, such that one or more of inner tubular body 14 and guide wire 18 may extend through deployment actuator reset member 44 in an axial direction. In some embodiments, when inner tubular body 14 is moved in corresponding direction with deployment actuator reset member 44 in either the direction of arrows X or X′, as described above, it will be appreciated that a corresponding movement of the guide wire 18 is not provided, because the guide wire 18 is not directly connected to deployment actuator reset member 44 in these embodiments.

Although actuator assembly 38 has been described to include a substantially mechanical structure including, for example, energy storage member 46 including a spring that cooperates with a selectively releasable fastener 42 that includes a latch, it will be appreciated that actuator assembly 38 is not limited to a particular structural implementation. As such, actuator assembly 38 may include other embodiments that include, for example, a substantially pneumatically operated device. Accordingly, in some embodiments of the present disclosure actuator assembly 38 may include a substantially pneumatically operated device that may include, for example, a vacuum or a source of fluid that is depressurized or pressurized (depending on whether a vacuum is used and/or the implementation of the fluid mechanism) such that the vacuum or fluid may be imparted to bore 24/cavity 36 and cause movement of the inner tubular body 14 in either the direction of arrow X or X′.

Referring to FIG. 2A, an enlarged view of the distal end 10b of an example embodiment of a revascularization device 10 of the present disclosure is shown. As shown, the distal end 10b may include an atraumatic tip 60 attached to a distal end 12b of the outer tubular body 12. Alternatively, it will be appreciated that the atraumatic tip 60 may be formed over and attached to an outer surface 62 of the outer tubular body 12.

In some embodiments, atraumatic tip 60 may include a tubular shaped-body having a blunt and/or dull distal end 60b. Further, one or more of an outer surface 64 of atraumatic tip 60 and an outer surface 62 of outer tubular body 12 may include a substantially thin, friction-reducing coating (not shown) that permits the distal end 10b to be easily moved within a vessel, V, such that any contact of the outer surfaces 62 and 64 with an inner surface, V_{I}, of vessel, V, does not impair movement of the distal end 10b in the direction of either arrow X or X′ within the vessel, V.

In some embodiments, the distal end 10b of revascularization device 10 may also include an inflatable portion 66 attached to, for example, the outer surface 62 of outer tubular body 12 and at least proximate to atraumatic tip 60. In some embodiments, inflatable portion 66 may be selectively inflated when, for example, the distal end 10b of revascularization device 10 is positioned proximate to a blockage, B, that is located within the bore, V_{B}, of the vessel, V. As seen in FIGS. 2C and 2D, when inflatable portion 66 is inflated, the inflatable portion 66 contacts the inner surface, V_{I}, of vessel, V, such that the distal end 10b of revascularization device 10 locates in a substantially axial center of vessel, V, and reduces the likelihood of the distal end 10b of revascularization device 10 becoming radially deviated from the axial center of the vessel, V, when, for example, the orientation of the inner tubular body 14 is forcefully deployed as described above and shown in FIG. 1B.

Methods for operating a revascularization device of the present disclosure are now described. As seen in FIG. 2A, the distal end 10b of revascularization device 10 may be navigated within the bore, V_{B}, of vessel, V, such that the atraumatic tip 60 is located proximate to a blockage, B, within the vessel, V. The blockage, B, may completely or partially occlude vessel V. “Partially occluded” vessels are defined as vessels with at least one blockage B that has one or more openings with the same or smaller diameter than the diameter of the inner tubular body 14. Then, as seen in FIG. 2B, a user may axially extend guide wire 18 out of bore 22 through distal end 14b of the inner tubular body 14 such that a distal end 18b of guide wire 18 may engage, but not penetrate, a substantially hard, proximal portion, B_{P}, of the blockage, B.

As seen in FIG. 2B, upon distal end 18b of guide wire 18 engaging the proximal portion, B_{P}, of blockage, B, distal end 18b of guide wire 18 may bend or curve such that a user may tactilely recognize the nature, physical characteristics, and/or location of blockage, B, within vessel V. Referring to FIG. 2C, the user may then retract guide wire 18 in the direction of the arrow X′ within the bore 22 of inner tubular member 14, such that the distal end 18b of guide wire 18 may be located/withdrawn into at least the bore 20 of outer tubular member 12. The distal end 18b of guide wire 18 may be withdrawn a short distance within bore 20, e.g., proximate to the distal end 12b of outer tubular member 12, or farther back in the direction of arrow X′ within bore 20 as necessary. In some embodiments, before, during, or after the retraction of guide wire 18 as described above, a user may inflate inflatable portion 66, as described above and as seen, e.g., in FIG. 2C. Further, a user may deflate portion 66 to reposition or retract revascularization device 10.

Referring to FIG. 2D, once guide wire 18 and inflatable portion 66 have been manipulated as described above, manipulation of the orientation of the inner tubular member 14 may be carried out to deploy inner tubular member 14, as described above and shown, for example, in FIG. 1B. As shown in FIG. 2D, upon causing the forceful, plunging deployment of inner tubular body 14 according to the direction of the arrow, X, the distal end 14b of inner tubular body 14 may be forcefully plunged into and/or through the proximal portion, B_{P}, of the blockage, B, which may include a relatively less hard, intermediate portion, B_{I}, of the blockage, B, and a relatively hard distal portion, B_{D}, of the blockage, B, and may plunge through blockage B in a single, plunging action.

As illustrated in FIG. 2D, in some embodiments portions and/or particulates of the blockage, B, may be aspirated into bore 22 of inner tubular body 14 during and/or after the penetration of the blockage, B, as discussed in more detail below. Once the blockage, B, has been penetrated by inner tubular body 14, a passage, bore, B_{B} (see, e.g., FIG. 3A), or the like may be formed in the blockage, B, in order to provide revascularization (i.e., relieve a complete obstruction within the vessel, V, in order to permit blood flow) or improved flow (i.e., widen a constricted bore or passage in a partial obstruction) of the vessel, V. Once the bore, B_{B}, in the blockage, B, has been formed, inner tubular body 14 may be moved in the direction of the arrow, X′, as described above and shown, for example, in FIGS. 1A and 1B.

In some embodiments of methods of the present disclosure, the blockage, B, may be characterized by what is referred to among cardiovascular technicians as a chronic total occlusion (CTO) or total coronary occlusion. Procedural success for treating a totally occluded vessel, V (e.g., a coronary artery), is usually determined by the age of the blockage, B. For example, a CTO is typically identified as a complete blockage (i.e., substantially no blood flow) within the vessel, V, that has been in existence for about three or more months (i.e., in some circumstances, a CTO may exist within a vessel, V, for more than a year).

Many CTO blockages consist of a homogenous or non-homogenous rock-hard, calcified, long lesion. As measured axially along the length of a vessel, V, a CTO blockage, B, may include a length, B_{L}, that may be as much as, for example, about 20 millimeters (mm). As such, it will be appreciated that inner tubular body 14 of revascularization device 10 may be provided with the capability to impart adequate plunging force (arising from, e.g., the physical characteristics of energy storage member 46) in the direction of the arrow, X, such that the distal end 14b of inner tubular body 14 may penetrate and/or pierce through substantially the entire length, B_{L}, of the CTO blockage, B, without being: reciprocated (i.e., moving in a “jackhammering” fashion), spun or twisted (i.e., moving in a drilling fashion), or oscillated (i.e., ultrasonically vibrated). It will be appreciated that “jackhammering”, drilling, and/or oscillating of a CTO blockage, B, during a revascularization attempt could undesirably result in a decreased likelihood of a successful procedure, for example, by piercing the vessel, V, or, by undesirably introducing a significant amount of loosened blockage particulates into the bore, V_{B}, of the vessel, V. Further, it will be appreciated that undesirable “jackhammering”, drilling, and/or oscillating movements would greatly slow down revascularization procedures, possibly needing multiple revascularization attempts and potentially causing medical professionals to need as much as five to thirty minutes to revascularize a vessel. In contrast, the revascularization device 10 of the present disclosure may more efficiently revascularize a completely or partially occluded vessel, V, within a significantly shorter period of time.

In some embodiments of the device 10 of the present disclosure, the distal end 14b of inner tubular body 14 may further include a piercing portion 68, which may aid in penetrating a vessel blockage, B. In some embodiments, piercing portion 68 may include a bladed structure. In some embodiments, piercing portion 68 may include, for example, a circumferential, zigzag or saw-tooth structure. It will be appreciated, however, that piercing portion 68 is not limited to a bladed, zigzag or saw-tooth structure and may include any desirable configuration. In some embodiments, piercing portion 68 may functionally assist in penetrating a CTO blockage, B, although the distal end 14b of inner tubular body 14 may be provided with a non-bladed structure in some embodiments. In some embodiments, piercing portion 68 may functionally assist in penetrating a partial occlusion and/or total occlusion.

In some embodiments of the present disclosure, a distal end 14b of inner tubular body 15, either with or without a piercing portion 68, may contain at least one distal opening in order to permit loosened blockage particulates to be aspirated into bore 22 of inner tubular body 14. In some embodiments, as shown in FIG. 2D, a vacuum source, S, may be in communication with bore 22 of inner tubular body 14 in order to assist in the evacuation of blockage particulates from within the vessel, V. Further, as seen in FIGS. 1A and 1B, a seal 70 may be arranged within the bore 20 of outer tubular body 12 and located adjacent to an inner surface 72 of outer tubular body 12 and an outer surface 56 of inner tubular body 14 to prevent fluids or blockage particulates to be aspirated into bore 24/cavity 36 of actuator portion 16.

In some embodiments of the present disclosure, revascularization device 10 may be modular, such that medical interventional devices suitable for additional vascular procedures may be put in place of inner tubular body 14. An example embodiment of a medical interventional device 100 for implanting a stent 104 is shown in FIGS. 3A-3D. In some embodiments, an inner tubular body 14 module may be withdrawn in the direction of the arrow, X′, and detached from the revascularization device 10 in order to permit attachment of a medical interventional device 100 module to the revascularization device 10. Similarly, a medical interventional device 100 module attached to revascularization device 10 may be replaced with an inner tubular body 14 module or another medical interventional device module.

In some embodiments, at least a portion of the outer surface 56 of inner tubular body 14 may be detachably affixed to a portion 54 of actuator assembly 38. In some embodiments, at least a portion of the outer surface 56 of inner tubular body 14 may be detachably affixed to a surface of deployment actuator reset member 44. Similarly, in some embodiments, at least a portion of the outer surface of medical interventional device 100 may be detachably affixed to a portion 54, of actuator assembly 38. In some embodiments, at least a portion of the outer surface medical interventional device 100 may be detachably affixed to a surface of deployment actuator reset member 44.

In some embodiments, inner tubular body 14, medical interventional device 100 and/or actuator assembly 38 may include latches and/or corresponding grooves which engage each other when inner tubular body 14 or medical interventional device 100 is slid into actuator assembly 38. In some embodiments, for example, a portion of the outer surface 14b of inner tubular body 14 and medical interventional device 100, as well as a portion 54 of the inner surface of actuator assembly 38 may include threads and/or corresponding grooves, which allow for attachment using a screw-like rotational motion. It will be appreciated that other means well known in the art may also be used to attach and detach inner tubular body 14 or medical interventional device 100 and revascularization device 10,

In some embodiments, as shown, for example, in FIGS. 3A-D, a medical interventional device 100 module may include, for example, a distal end 100b including an inflatable portion 102 that carries a stent 104. Embodiments of methods of the present disclosure of using a medical interventional device 100 module including an inflatable portion 102 that carries a stent 104 are now described.

As seen in FIGS. 3A-3B, for example, the orientation of the medical interventional device 100 may be manipulated such that a distal end 100b including the inflatable portion 102 and stent 104 may be located within a bore, B_{B}, which may have been created in blockage, B, by use of an embodiment of revascularization device 10 that includes inner tubular body 14. Manipulation of distal end 100b may be accomplished by manipulation of deployment actuator 40 as described above (see FIGS. 1A-1B). Then, as seen in FIG. 3C, the orientation of medical interventional device 100 may be further manipulated such that inflatable portion 102 and stent 104 are correspondingly expanded in order to increase and/or maintain the dimensions of bore, B_{B}, of blockage, B. Then, as seen in FIG. 3D, the orientation of the medical interventional device 100 may be further manipulated such that the distal end 100b including inflatable portion 102 is inflatably or deflatably retracted (such that the stent 104 is left within the bore, B_{B}, of the blockage, B) in order to permit medical interventional device 100 to be withdrawn from within the bore, V_{B}, of vessel, V.

The present invention has been described with reference to certain example embodiments thereof. However, it will be readily apparent to those skilled in the art that it is possible to embody the invention in specific forms other than those of the example embodiments described above. This may be done without departing from the spirit of the invention. The example embodiments are merely illustrative and should not be considered restrictive in any way. The scope of the invention is defined by the appended claims and their equivalents, rather than by the preceding description.

What is claimed is:

1. A medical device, comprising:
an actuator portion;
an inner tubular body connected to the actuator portion;
an outer tubular body connected to the actuator portion, wherein the inner tubular body is disposed within a bore of the outer tubular body; and
a guide wire extending through one or more of the actuator portion, inner tubular body and outer tubular body.

2. The medical device according to claim 1, wherein the actuator portion provides
means for manipulating an orientation of the inner tubular body relative to one or more of the outer tubular body and guide wire for penetrating a blockage within a vessel for revascularizing the vessel.

3. The medical device according to claim 2, wherein the manipulation of the orientation of the inner tubular body is a single, forceful plunging movement through the blockage, wherein the blockage includes a chronic total occlusion or partial occlusion, and wherein the vessel includes an artery.

4. The medical device according to claim 1, wherein the inner tubular body includes a distal end that provides
means for penetrating a blockage within a vessel for revascularizing the vessel in response to a manipulation of the actuator portion.

5. The medical device according to claim 4, wherein the penetrating the blockage within the vessel is conducted by a single, forceful plunging movement of the inner tubular body through the blockage, wherein the blockage includes a chronic total occlusion or partial occlusion, and wherein the vessel is an artery.

6. The medical device according to claim 4, wherein a bore of the inner tubular body is connected to an aspiration device.

7. The medical device according to claim 4, wherein a distal end of the inner tubular body includes a piercing portion.

8. The medical device according to claim 7, wherein the piercing portion forms an opening at the distal end of the inner tubular body, and wherein a bore of the inner tubular body is connected to an aspiration device for aspirating a portion of the blockage out of the vessel.

9. The medical device according to claim 1, wherein the actuator portion includes
a deployment actuator,
a selectively releasable fastener connected to the deployment actuator,
a deployment actuator reset member connectable with the selectively releasable fastener, and
an energy storage member connected to the deployment actuator reset member.

10. The medical device according to claim 9, wherein the deployment actuator member includes a depressible button, wherein the selectively releasable fastener includes a latch, wherein the deployment actuator reset member includes a lever, wherein the energy storage member includes a spring.

11. The medical device according to claim 9, wherein the distal end of the inner tubular body further comprises a piercing portion.

12. The medical device according to claim 11, further comprising an inflatable portion connected to an outer surface of the outer tubular body.

13. The medical device according to claim 1, wherein the inner tubular body is detachably connected to the actuator portion.

14. The medical device according to claim 1, further comprising an inflatable portion.

15. A method of revascularizing a totally occluded or partially occluded vessel containing at least one blockage, comprising:
providing a revascularization device comprising
an actuator portion;
an inner tubular body connected to the actuator portion, wherein the inner tubular body comprises a distal end;
an outer tubular body connected to the actuator portion, wherein the inner tubular body is disposed within a bore of the outer tubular body; and
a guide wire extending through one or more of the actuator portion, inner tubular body and outer tubular body.

16. The method of claim 15, further comprising
locating the distal end of the inner tubular body proximate to the at least one blockage in the vessel; and
manipulating the actuator portion so that the distal end of the inner tubular body penetrates the blockage.

17. The method of claim 16, wherein the revascularization device further comprises an inflatable portion, and wherein the method further comprises inflating the inflatable portion.

18. A method of revascularizing a totally occluded or partially occluded vessel, comprising:
providing a revascularization device comprising
an actuator portion;
an inner tubular body detachably connected to the actuator portion, wherein the inner tubular body comprises a distal end;
manipulating the actuator portion so that the distal end of the inner tubular body penetrates the blockage, creating a bore through the blockage;
detaching the inner tubular body; and
attaching a medical interventional device to the actuator portion.

19. The method of claim 18, wherein the medical interventional device comprises a stent attached thereto; and the method further comprises placing the stent at least partially within the bore through the blockage by manipulating the actuator portion.

20. The method of claim 18, wherein the revascularization device further comprises an inflatable portion, and the method further comprises inflating the inflatable portion.
